(19) United States
(12) Patent Application Publication
(10) Pub. No.: US 2013/0079249 A1
(43) Pub. Date: Mar. 28, 2013

(54) GLYCOSYLATION ASSAY, GLYCOANALYSIS ARRAY AND AN ASSAY SYSTEM

(71) Applicants: Albena SAMOKOVLISKY, Ashdod (IL); Yehudit AMOR, Jerusalem (IL); Yeshayahu YAKIR, Rishon Le Zion (IL); Chanan HIMMELFARB, Nof-Ayalon (IL); David DABOUSH, Moshav Zilan (IL); Ilana BELZER, Rishon Le Zion (IL); Iris LIEDER, Tel Aviv (IL); Joseph COHEN, Givat Shmuel (IL)

(72) Inventors: Albena SAMOKOVLISKY, Ashdod (IL); Yehudit AMOR, Jerusalem (IL); Yeshayahu YAKIR, Rishon Le Zion (IL); Chanan HIMMELFARB, Nof-Ayalon (IL); David DABOUSH, Moshav Zilan (IL); Ilana BELZER, Rishon Le Zion (IL); Iris LIEDER, Tel Aviv (IL); Joseph COHEN, Givat Shmuel (IL)

(21) Appl. No.: 13/596,137

(22) Filed: Aug. 28, 2012

Related U.S. Application Data
(60) Provisional application No. 61/532,559, filed on Sep. 9, 2011.

(51) Int. Cl. G01N 33/68

(52) U.S. Cl. CPC G01N 33/6854
USPC 506/18; 506/39

(57) ABSTRACT

An improved glycosylation assay, glycoanalysis array and an assay system for performing glycosylation assays glycoanalysis array.

This application claims priority from U.S. Provisional Application No. 61/532,559, filed on 9 Sep. 2011, which is hereby incorporated by reference as if fully set forth herein.

### FIELD OF THE INVENTION

The present invention relates to an improved glycosylation assay.

### BACKGROUND OF THE INVENTION

Oligosaccharides and polysaccharides are polymers that consist of monosaccharide (sugar) units, connected to each other via glycosidic bonds. These polymers have a structure that can be described in terms of the linear sequence of the monosaccharide subunits, which is known as the two-dimensional structure of the polysaccharide. Polysaccharides can also be described in terms of the structures formed in three dimensions by their component monosaccharide subunits.

The saccharide chain has, like a chain of DNA or protein, two dissimilar ends. In the case of saccharide chains, these are the reducing end (corresponding to the aldehyde group of the linear sugar molecule) and the non-reducing end. Unlike proteins and DNA, however, polysaccharides are generally branched, with essentially each of the sugar units in the polysaccharide serving as an optional branching point.

There are a number of proteins that bind to saccharides. Many of these proteins bind specifically to a certain short mono or disaccharide sequence. Lectins are a broad family of proteins that bind saccharides. A large number of plant lectins have been characterized and are used in research. Many mammalian lectins have also been characterized. Antibodies are proteins that specifically recognize certain molecular structures. Antibodies may also recognize saccharide structures, as do lectins. Glycosidases are enzymes that cleave glycosidic bonds within the saccharide chain. Also glycosidases may recognize certain oligosaccharide sequences specifically. Glycosyltransferases are enzymes that transfer a sugar unit to acceptor molecules. In vivo, these acceptor molecules are the growing glycan structures.

The structural determination of polysaccharides is of fundamental importance for the development of glycobiology. Research in glycobiology relates to subjects as diverse as the bacterial cell walls, blood glycans, to growth factor and cell surface receptor structures involved in viral disease, such as HIV infection, autoimmune diseases such as insulin-dependent diabetes and rheumatoid arthritis, and abnormal cell growth as it occurs in cancer.

The importance of glycomolecules is highlighted by the discovery of penicillin, an inhibitor of glycomolecule synthesis in the bacterial cell-wall and possibly the most successful antibiotic discovered to date.

Another example is the medical use of heparin, a glycosaminoglycan that inhibits blood clotting and is today widely used in medicine. Further examples of medically-important glycomolecules include: glycosaminoglycans (GAGs), heparan sulphate, monoclonal antibodies, cytokines (e.g. IL-8, TNF, and the blockbuster EPO), chemokines (e.g. acidic fibroblast growth factor) and various growth factors. The aforementioned cytokines, chemokines and growth factors are also capable of binding to GAGs and other polysaccharides, and therefore may be also be considered to be lectins.

The structural complexity of polysaccharides has hindered their analysis. For example, saccharides are believed to be synthesized through a template-independent mechanism. In the absence of structural information, the researcher must therefore assume that the building units are selected from any of the saccharide units known today. In addition, these units may have been modified, during synthesis, e.g., by the addition of sulfate groups. Without the ability to measure such carbohydrate structural information, the researcher cannot determine the true, correct glycosylation pattern for populations of cells, for example in a tissue. In addition, these units may have been modified, e.g. by the addition of sulfate groups, during synthesis, such that merely understanding which types of saccharides may have been added does not provide a complete picture.

Furthermore, the connections between saccharide units are multifold. A saccharide may be connected to any of the C1, C2, C3, C4, or C6 atoms if the sugar unit to which it is connected is a hexose. Moreover, the connection to the C1 atom may be in either alpha or beta configuration. In addition, the difference in structure between many sugars is minute, as a sugar unit may differ from another merely by the position of the hydroxyl groups (epimers).

In vivo, glycosylation is tissue dependant and can vary significantly with cell state. In vitro, glycosylation strongly depends on growth conditions: the type of cell, nutrient concentrations, pH, cell density, and age can affect the glycosylation patterns of glycoproteins. The number of glycoforms and their relative abundance within a cell are affected by the intrinsic structural properties of the individual protein, as well as the repertoire of glycosylation enzymes available (including their type, concentration, kinetic characteristics, compartmentalization). This repertoire has been shown to change upon changes in cell state (e.g. oncogenic transformation).

### SUMMARY OF THE INVENTION

The present invention provides an improved glycosylation assay. The improved assay is able to provide more accurate results. Examples of these improvements include but are not limited to assay optimization improvements to the assay system; the K fingerprint arraying format which relates to improvements in the array; QC monitors and calibration, which relate to improvements in the assay system; and implementation of an out of assay flag for the array and the system.

According to at least some embodiments, there is provided a glycoanalysis array, comprising a planar substrate and a plurality of saccharide binding agents present on a surface of said substrate at a plurality of predetermined locations, each of said plurality of saccharide binding agents being present at a plurality of separate predetermined locations on said surface, wherein said plurality of separate predetermined locations relates to a plurality of concentrations of said saccharide binding agent at said locations in a concentration curve; said planar substrate being adapted for being contacted with a sample comprising a glycoprotein, such that said glycoprotein binds specifically to at least one saccharide binding agent and forms a detectable binding complex, such that a baseline for non-specific binding is determined according to said concentration curve.

According to at least some embodiments, there is provided a glycoanalysis array, characterized in that comprising: a planar substrate; and a plurality of contact portions present on a surface of said planar substrate at a plurality of predetermined locations, a plurality of saccharide binding agents are provided in the plurality of contact portions (10), the plurality of saccharide binding agents present in predetermined locations on said planar substrate, each of said plurality of saccharide binding agents is present at a plurality of separate predetermined locations on said surface, wherein said plurality of separate predetermined locations are provided with a plurality of concentrations of said saccharide binding agent in a concentration curve; said planar substrate is provided to contact with a sample comprising a glycoprotein, such that said glycoprotein binds specifically to at least one saccharide binding agent at the plurality of contact portions and forms a detectable binding complex, such that a baseline for non-specific binding is determined according to said concentration curve.

Optionally, said contact portions is selected from grooves, indentations or wells in shape of circular, elliptic, rectangular.

Optionally said detectable binding complex is detectable through a binding signal and wherein said binding signal is linear over at least five of the total number of saccharide binding agent concentrations in said concentration curve. Optionally said planar substrate features indentations or wells. Optionally said planar substrate comprises at least one of a membrane, glass or plastic. Optionally said planar substrate is derivatized.

Optionally said array further comprises a plurality of labeled predetermined locations on said planar substrate for providing a high signal for supporting image analysis of said array after being contacted with said sample. Optionally said array further comprises a plurality of saccharide binding agents as calibration standards in predetermined locations on said planar substrate. Optionally said planar substrate is divided to a plurality of pads and wherein each pad comprises a separate plurality of calibration standards.

Optionally said saccharide binding agents comprise lectins or antibodies, or modified components or a combination thereof.

Optionally a concentration range for each lectin on said substrate is from 0.01 mg/ml to 10 mg/ml.

Optionally said concentration range is 0.001 mg/ml to 10 mg/ml.

Optionally said concentration range is from 0.01 mg/ml to 5 mg/ml.

Optionally each location has a spot size diameter and wherein said spot size diameter is in a range of from 0.05 mm to 75 mm.

Optionally each location has a spot size diameter and wherein said spot size diameter is in a range of from 80 microns to 2500 microns.

Optionally said glycoprotein comprises an IgG antibody or fragment.

Optionally said IgG amount is in the range of 1.8-12 μg per location.

Optionally said IgG concentration is in a range of from 0.001 micro-molar to 100 micro-molar in a solution for contacting said IgG antibody or fragment to said saccharide binding agents.

Optionally said solution comprises a detergent for unfolding said IgG antibody or fragment and exposing at least one glycan.

According to at least some embodiments of the present invention, there is provided an assay system for performing glycosylation assays with a plurality of arrays as described herein, comprising a kit for performing said glycosylation assay, a detector for detecting said detectable binding complex through a binding signal to obtain binding data and an assay optimization module for comparing reference data and said binding data obtained with the sample glycoprotein, to determine suitable assay conditions by calculation of optimal lectin activity, measurement of lectins that generate signals only with the sample glycoprotein and calculation of the slide background value compared to a calculated baseline defined based on an experimentally determined database created using a wide range of sample types.

According to at least some embodiments, there is provided an assay system for performing glycosylation assays, characterized in that comprising:
a plurality of arrays, each of the plurality of array comprise
   a planar substrate; and
   a plurality of contact portions present on a surface of said planar substrate at a plurality of predetermined locations, a plurality of saccharide binding agents are provided in the plurality of contact portions, each of said plurality of saccharide binding agents is present at a plurality of separate predetermined locations on said surface, wherein said plurality of separate predetermined locations are provided with a plurality of concentrations of said saccharide binding agent in a concentration curve; said planar substrate is provided to contact with a sample comprising a glycoprotein, such that said glycoprotein binds specifically to at least one saccharide binding agent at the plurality of contact portions and forms a detectable binding complex, such that a baseline for non-specific binding is determined according to said concentration curve;
an assay performance module, the plurality of arrays are connected to the assay performance module, or contained in the assay performance module;
a detector connected to said assay performance module, a binding of the saccharide binding agents to the sample protein is detected through said detector; said detector is in communication with assay data analyzer or connected to assay data analyzer;
an assay data analyzer, which is in communication with said detector or connected to said detector;
an out of assay flag module connected to said assay data analyzer or in communication with said assay data analyzer; and
   an assay optimization module, connected to said assay data analyzer or in communication with said assay data analyzer.

Optionally, said contact portions is selected from grooves, indentations or wells in shape of circular, elliptic, rectangular.

Optionally, said arrays is inserted into or provided to said assay performance module, optionally only once for assay optimization.

Optionally, said detector is combined with or separated from said assay performance module.

Optionally, said detector is combined with or alternatively separate from said assay data analyzer.

Optionally, said assay optimization module comprises a database containing data relating to binding determined under different experimental conditions for the protein of interest.

Optionally, said assay optimization module receives a file containing optimized experimental results for other proteins of a particular type.

Optionally said assay optimization module further determines suitable assay conditions according to one or more of the following parameters: binding time, temperature and pH value of buffer for incubating the sample protein with the saccharide binding agents; saccharide binding agent signal, signal ratio, a set of designated saccharide binding agents that react when the exposure was not optimal, background value and background value compared to the calibration standard background; detergent concentration in the sample buffer; and sample protein concentration.

Optionally said assay optimization module optimizes sample glycoprotein concentration, substrate format and assay format.

Optionally said sample glycoprotein comprises an IgG antibody and wherein said assay optimization module determines whether the IgG sample antibody has Fab glycosylation or O-link glycosylation, such that specific optimization conditions are implemented and a warning is issued regarding the presence of such glycosylation.

Optionally said assay optimization module determines an amount of exposure solution to be applied to said sample glycoprotein, wherein said exposure solution features a detergent selected from the group consisting of cholate, deoxycholate, C16TAB, LysoPC, CHAPS, Zwittergent, Octylglucoside, Digitonin, Lubrol, C12E8, Triton X-100, Nonidet P-40 SDS (sodium dodecyl sulfate), and Tween-80, at a percentage known in the art for unfolding proteins.

Optionally said assay optimization module determines a condition matrix relating to one or more of the following: Exposure solution concentration optimization: 0.001 to 1%, Temperature optimization: 50-80° C., Time optimization of pre-treatment incubation: 1 minute to 1.

Optionally the system further comprises at least one QC (quality control) monitor selected from the group consisting of spots evaluation by homogeneity of foreground, homogeneity of background, similarity between mean median density, level of saturation; array validation to evaluate a quality of an entire planar substrate according to background of the array, control spots, intra array reproducibility; evaluation of normalization between sample and calibration locations; determination of overall array signal.

Optionally the system further comprises a calibration protein for being applied to a plurality of predetermined locations containing said saccharide binding agents as a calibration sample, to calibrate saccharide binding agent reactivity according to a golden fingerprint standard by said assay optimization module.

Optionally said assay optimization module determines an out of assay flag according to a comparison of binding signals from said sample glycoprotein to said calibration sample.

Optionally said assay optimization module issues a warning according to said out of assay flag.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. All publications, patent applications, patents, and other references mentioned herein are incorporated by reference in their entirety. These include, but are not limited to, WO00/68688 and WO01/84147 (US20060194269, US20070092915, U.S. Pat. No. 7,056,678 and U.S. Pat. No. 7,132,251), WO02/37106 (US20040132131), and WO02/44714 (U.S. Pat. No. 7,079,955 and US20040153252). In the case of conflict, the present Specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 shows an exemplary array according to at least some embodiments of the present invention;

FIG. 2 shows an exemplary system according to at least some embodiments of the present invention;

FIG. 3 relates to exemplary printing concentrations for an exemplary lectin according to at least some embodiments of the present invention;

FIG. 4 shows an exemplary QC and calibration process for the operation of the system of FIG. 2; and

FIG. 5 shows results of the assay optimization experiment layout, samples and array types in combination with the different tested sample exposure conditions, the experiment which was tested for one array type (one pad slides or multi pad slide) using an exemplary IgG antibody, Avastin through the above system.

### DESCRIPTION OF THE PREFERRED EMBODIMENTS

The present invention is of an improved glycosylation assay. According to preferred embodiments of the present invention, such an assay may optionally and preferably be performed according to U.S. Pat. No. 7,056,678, owned in common with the present application, hereby incorporated by reference as if fully set forth herein. For example, this patent describes a method for the structural analysis of a saccharide, comprising: providing on a surface a plurality of essentially sequence-specific and/or site-specific binding agents; contacting the surface with a mixture of saccharides to be analyzed, for example an extract of glycomolecules from specific compartments of cells or tissue washing or otherwise removing unbound saccharide or saccharide fragments; adding to the surface obtained previously an essentially sequence- and/or site-specific marker, or a mixture of essentially sequence- and/or site-specific markers; acquiring one or more images of the markers that are bound to the surface; and deriving information related to the identity of the saccharide being analyzed from the image.

In one aspect, the present invention is to provide a glycoanalysis array 102, characterized in that comprising: a planar substrate 1; and a plurality of contact portions 10 present on a surface of said planar substrate 1 at a plurality of predetermined locations, a plurality of saccharide binding agents are provided in the plurality of contact portions 10, the plurality of saccharide binding agents act as detectors of the glycan structure on the analyzed sample in predetermined locations on said planar substrate, each of said plurality of saccharide binding agents is present at a plurality of separate predetermined locations 10 on said surface, wherein said plurality of separate predetermined locations 10 are provided with a plurality of concentrations of said saccharide binding agent in a concentration curve; said planar substrate 1 is provided to contact with a sample comprising a glycoprotein, such that said glycoprotein binds specifically to at least one saccharide binding agent at the plurality of contact portions 10 and forms a detectable binding complex, such that a baseline for non-specific binding is determined according to said concentration curve.

In another aspect, the present invention is to provide an assay system 100 for performing glycosylation assays, characterized in that comprising: a plurality of arrays 102, each of the plurality of array 102 comprise: a planar substrate 1; and a plurality of contact portions 10 present on a surface of said planar substrate 1 at a plurality of predetermined locations, a plurality of saccharide binding agents is provided in the plurality of contact portions 10, each of said plurality of saccharide binding agents being present at a plurality of separate predetermined locations 10 on said surface, wherein said plurality of separate predetermined locations 10 are provided with a plurality of concentrations of said saccharide binding agent in a concentration curve; said planar substrate 1 is provided to contact with a sample comprising a glycoprotein, such that said glycoprotein binds specifically to at least one saccharide binding agent at the plurality of contact portions 10 and forms a detectable binding complex, such that a baseline for non-specific binding is determined according to said concentration curve; an assay performance module 104, the plurality of arrays 102 are connected to the assay performance module 104, or contained in the assay performance module 104; a detector 105 connected to the assay performance module 104, a binding of the saccharide binding agents to the sample protein is detected through the detector 105; the detector 105 is in communication with assay data analyzer 106; an assay data analyzer 106, which is in communication with the detector 105; an out of assay flag module 107 connected to the assay data analyzer 106 or in communication with the assay data analyzer 106; and an assay optimization module 108, connected to the assay data analyzer 106 or in communication with the assay data analyzer 106.

Optionally, said contact portion 10 is selected from grooves, indentations or wells in shape of circular, elliptic, rectangular.

Optionally, said array 102 is inserted into or provided to said assay performance module (104).

Optionally, said detector 105 is combined with or separated from said assay performance module 104.

Optionally, said detector 105 is combined with or alternatively separate from said assay data analyzer 106.

Optionally, said assay optimization module 108 comprises a database containing data relating to binding determined under different experimental conditions for the protein of interest.

Optionally, said assay optimization module 108 receives a file containing optimized experimental results for other proteins of a particular type.

The surface on which the binding agents are provided may comprise, for example, a bead or an array, or a multi-well plate (such as a 96 well plate for example), but preferably comprises a planar substrate. The array on the planar substrate (optionally featuring indentations or wells) may optionally comprise any of membranes, glass or plastic surfaces and the like.

Binding of the saccharide-binding markers may optionally be detected by acquiring images of the markers, and generating a map of recognition sites of the polysaccharide being analyzed, to derive partial structure distribution on the sample structure, and hence at least partial sequence information relating to the polysaccharide.

The markers may optionally comprise chromogenic binding agents, such that images are provided which are colors that develop on the surface of the substrate, through binding of the binding agents to the complex for example. Alternatively, the markers may be labeled binding agents, such that images of the markers are provided according to a signal from the label. Images may be acquired, for example, by the use of optical filters, laser scanners or by photographing and/or digitizing the images.

Additional methods and assays for determining a glycosylation pattern or “fingerprint” for a sample, such as for a cell or human IgG for example, are also disclosed in US Patent Application No. 20050186645, also owned in common with the present application, which is hereby incorporated by reference as if fully set forth herein. This application describes a method for obtaining information about the carbohydrate content of a glycomolecule by adding a glycomolecule to a substrate to which is attached one or more saccharide-binding agents (also referred to herein as first saccharide-binding agents). The first saccharide-binding agents that have bound the glycomolecule are identified, and the resulting binding information is used to generate a fingerprint of the glycomolecule.

The essentially sequence- and/or site-specific binding agents of the present invention may comprise, for example, lectins (such as colored lectins, fluorescent lectins, biotin labeled lectins) or antibodies (such as fluorescent antibodies, biotin-labeled antibodies, or enzyme-labeled antibodies). The method or assay may be performed using at least five lectins, such as, for example, 5, 10, 15, 20, 25, 30, 35, 40, 45 or 50 lectins, although optionally any number of lectins may be used, for example from about 5 lectins to about 100 or more lectins.

For example, the method may optionally be performed with a set of 20-30 lectins printed on a membrane-coated glass slide in replicates of 4-8 (or any other suitable set of replicates), or alternatively in a range of concentrations that provide a dose-response for each printed lectin. A sample of intact glycoprotein is applied to the array, and its binding pattern is detected by either direct labeling of the glycoprotein using any fluorophore, or by using a fluorophore-labeled probe that is directed at either the protein moiety—an antibody for example, or a carbohydrate moiety—a lectin. The resulting fingerprints are highly characteristic of the glycosylation pattern of the sample. The large number of lectins, each with its specific recognition pattern, ensures high sensitivity of the fingerprint to changes in the glycosylation pattern. Many fluorescent labels such as FITC, Rhodamine, Cy3, Cy5, or any of the Alexa dyes can be used. These fluorescent labels and dye labels are collectively termed herein “chromogenic labels”. In addition, labeling can be effected using biotin-avidin systems known in the art and/or with any other suitable type of label. Glycomolecules may optionally be modified before being analyzed as described above.

The method and assay of the present invention may optionally be carried out on whole cells. Alternatively, the method and assay may be carried out on a cell preparation (non-whole cell material), such as, for example, a membrane protein extract, a homogenized cell, or a crude membrane mixture.

In embodiments which comprise the use of a whole cell, the cell is preferably first fixed. For example, the cells may be fixed in suspension of RPMI culture medium by adding 1% glutaraldehyde in Sorenson's buffer, pH 7.3 (Tousimis Research Corp., Rockville, Md.), and washing in Sorenson's buffer after 24-48 hours (as described for example in Sanders et al, A high-yield technique for preparing cells fixed in suspension for scanning electron microscopy, The Journal of Cell Biology, Volume 67, 1975, pages 476 480).

Alternatively, cells may be fixed by immersing in PBS/3.7% formaldehyde for 60 minutes at ambient temperature, after which the cells are washed in distilled water (as described for example in Nimrichter et al, Intact cell adhesion to glycan microarrays, Glycobiology, vol. 14, no. 2; pp. 197-203, 2004).

Of course any type of cell fixation process may optionally be performed which permits detection of binding of saccharide-binding agents to the cells.

The method of the present invention may optionally and preferably be performed in vitro.

The method and assay of the present invention may optionally and preferably be carried out using the Qproteome Glycoprofiling Kit (Qiagen USA) or any GlycoScope type kit. Lectins used in such kits have been chosen by analysis of a set of over 80 lectins, using a large dataset of carefully chosen, well-characterized glycoproteins, and a large set of enzymatically synthesized glycovariants of these proteins. The lectins on the array are grouped according to their monosaccharide specificities, in cases where possible; lectins in the group that is denoted “complex” do not bind monosaccharides, but bind complex N-linked glycans. The groups and differences between lectins within each group are detailed below.

### Complex

The lectins in this group recognize branching at either of the two a-mannose residues of the tri-mannosyl core of complex N-linked complex glycans. Some of the lectins of this group are sensitive to different antennae termini as they bind large parts of the glycan structure. The lectins denoted Complex(1) and Complex(4) have a preference for 2,6-branched structures; lectin Complex(3) has a preference for 2,4-branched structures, and lectin Complex(2) recognizes with similar affinity both structures.

### GlcNAc

The lectins in this group bind N-acetylglucosamine (GlcNAc) and its β4-linked oligomers with an affinity that increases with chain length of the latter. The carbohydrate-specificity of both lectins in this group do not differ, yet differences in their binding patterns are observed and probably stem from the non-carbohydrate portion of the samples.

### Glc/Man

This group of lectins is a subgroup of the mannose binding lectins (see below), and are denoted Glc/Man binding lectins since they bind, in addition to mannose, also glucose. All of the lectins in this group bind to bi-antennary complex N-lined glycans with high affinity. In comparison to their affinity for bi-antennary structures, lectins Glc\Man(1) and (2) bind high mannose glycans with lower affinity, whereas lectin Glc\Man(3) will bind high mannose glycans with higher affinity.

### Mannose

This group consists of lectins that bind specifically to mannose. These lectins will bind high mannose structures and, with lower affinity, will recognize the core mannose of bi-antennary complex structures.

### Terminal GlcNAc

This lectin specifically recognizes terminal GlcNAc residues.

### Alpha Gal

These lectins bind terminal a-galactose (a-Gal). Lectin Alpha-Gal(1) binds both a-galactose and a-GalNAc (a-N-acetylgalactosamine) and may bind to both N and O-linked glycans. Lectin Alpha-Gal(3) binds mainly the Galili antigen (Gala1-3Gal) found on N-linked antennae.

### Beta Gal

These lectins specifically bind terminal (non-sialylated) β-galactose residues.

### Gal/GalNAc

These lectins are specific for terminal galactose and N-acetyl-galactoseamine residues. The different lectins within this group differ in their relative affinities for galactose and N-acetyl-galactoseamine.

Lectins (2) and (5) from this group bind almost exclusively Gal; lectins (1), (3) and (4) bind almost exclusively GalNAc. The relative affinities for GalNAc/Gal for the remaining lectins in the group are ranked: (8)>(7)>(6).

### Fucose

Lectins from this group bind fucose residues in various linkages.

Lectin Fucose(6) binds preferentially to 1-2-linked fucose; Lectin Fucose(8) binds preferentially to 1-3 and 1-6 lined fucose; Lectins Fucose(12) and (13) bind preferentially to Fuc1-4GlcNAc (Lewis A antigens).

These lectins generally do not bind the core fucose of N-linked oligosaccharides on intact glycoproteins due to steric hindrance.

### Sialic Acid

The sialic acid lectins react with charged sialic acid residues. A secondary specificity for other acidic groups (such as sulfation) may also be observed for members of this group.

Lectin Sialic Acid(1) recognized mainly 2-3-linked sialic acid; Lectin Sialic Acid(4) recognizes mainly 2-6-linked sialic acid.

It should be understood that these examples for methods and assays for detecting glycosylation are provided for the purposes of discussion only and are not intended to be limiting in any way, as any other suitable method and/or assay could optionally be used with the present invention.

The principles and operation of the present invention may be better understood with reference to the drawings and the accompanying description, as well as the following examples.

### Example 1

### Assay Optimization Improvements to the Assay System

This Example relates to assay optimization improvements to the assay system. The assay system is generally useful for examining glycosylation for many different types of proteins. However, specific improvements to the system have been discovered for one type of protein, which preferably relate to antibodies and preferably human IgG antibodies. Glycoanalysis of these proteins has surprisingly been found to require exposure of the glycans hidden in the protein structure, which is achieved through specific improvements to the glycoanalysis system. The system defines the optimal exposure conditions and takes the user through the optimization process including assay parameters and analysis parameters.

The system adjusts the array and the associated assay conditions for performing the assay with the adjusted array, and including optimal exposure temperature and exposure solution concentration in the sample buffer, the optimization protocol and software. The system preferably verifies that the optimal exposure treatment selected enables accurate glycoanalysis as compared to reference results obtained by traditional methods e.g. HPLC and/or through controls as described in greater detail below. The protein is optionally digested before being analyzed through HPLC or other assays, although it is preferably analyzed whole for the assay system described with regard to FIG. 2. The protocol can be used with or without HPLC reference to allow users to perform the calibration in house and in a simple manner. The system preferably features assay software and related protocol and samples.

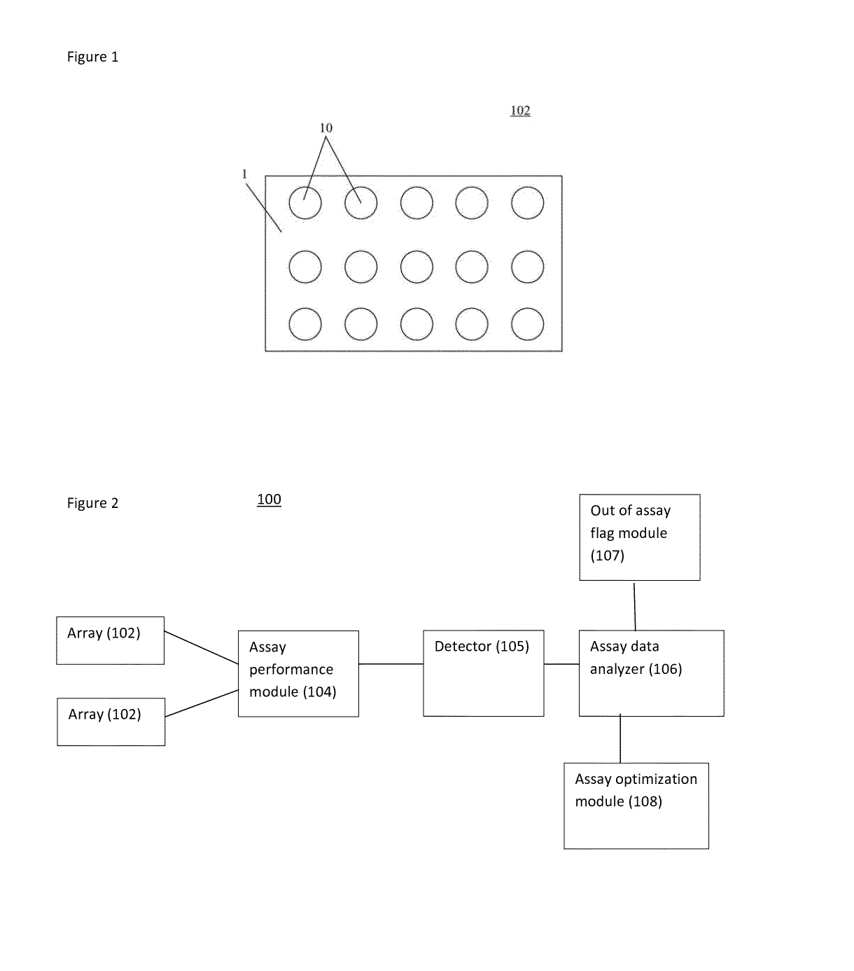

As shown in FIG. 1, a glycoanalysis array 102 of the present invention comprise: a planar substrate 1; and a plurality of contact portions 10 present on a surface of said planar substrate 1 at a plurality of predetermined locations, a plurality of saccharide binding agents is provided in the plurality of contact portions 10, the plurality of saccharide binding agents act as saccharide and glycan detectors according to their binding specificity in predetermined locations on said planar substrate 1, each of said plurality of saccharide binding agents being present at a plurality of separate predetermined locations 10 on said surface, wherein said plurality of separate predetermined locations 10 are provided with a plurality of concentrations of said saccharide binding agent in a concentration curve; said planar substrate 1 is provided to contact with a sample comprising a glycoprotein, such that said glycoprotein binds specifically to at least one saccharide binding agent at the plurality of contact portions 10 and forms a detectable binding complex, such that a baseline for non-specific binding is determined according to said concentration curve.

As shown in FIG. 2, an assay system 100 is provided for performing glycosylation assays. Assay system 100 features a plurality of arrays 102, each comprising a solid (preferably planar) substrate on which a plurality of sequence or site specific saccharide binding agents have been provided in a predetermined order (for a non-limiting example of the format for such an order, see Example 2 below). Such binding agents include but are not limited to antibodies, lectins and the like. Non-limiting examples of such lectins were described previously. The solid substrate may optionally comprise a porous membrane, such as a nitrocellulose for example, or a non-porous substrate, such as glass for example (the latter type of substrate is preferably derivatized to permit proteins to be securely bound thereto, as is known in the art). Array 102 may optionally be prepared according to the K fingerprint array format as described with regard to Example 2 below.

The arrays 102 are preferably treated through components of a kit based on the assay protocol and specific instructions in the assay optimization protocol, for example including one or more washing and/or blocking buffers as described herein. Arrays 102 may optionally inserted into (or otherwise provided to) an assay performance module 104 for automated preparation and performance of the assay protocol as described herein. Within assay performance module 104, sample proteins are applied to each array 102, followed by one or more washing steps. Optionally, before application of the sample proteins, one or more blocking, equilibration and/or washing steps are also performed. By “blocking” it is meant that a buffer is applied to block non-specific interactions. Such blocking is also optionally performed after the sample protein is applied to array 102. Arrays 102 are also referred to below as “slides”.

After application of the sample proteins and optionally after one or more washing steps, binding of the saccharide binding agents to the sample protein is detected through a detector 105. It should be noted that optionally the sample protein is bound first to the array 102, without the saccharide binding agents, and that the saccharide binding agents are applied afterward, but in any case the formation of complexes between the saccharide binding agent(s) and the sample protein is detected through detector 105. Detector 105 may optionally be combined with or alternatively may be separate from assay performance module 104.

Detector 105 preferably receives a raw signal that is directly indicative of binding; for example, if colorimetric reporters are used to detect formation of a binding complex, optionally detector 105 performs image analysis to obtain the raw binding signal. Optionally and preferably, an assay data analyzer 106 then analyzes the data to determine whether binding actually occurred. Detector 105 is in communication with assay data analyzer 106 but may optionally be combined with or alternatively separate from assay data analyzer 106.

Binding is measured by calculation of saccharide binding agent signals, such as lectin signals for example, and by calculation of the lectin signal ratio. For example, optionally calibration results or QC (quality control) factors may be considered to determine whether binding occurred, as described with regard to some of the Examples below. Optionally, as described with regard to Example 4, an out of assay flag module 107 flags any unusual or “outlier” results and also detect assay related problems; these results may optionally be eliminated from further consideration, also as described in greater detail below.

An assay optimization module 108 then receives the binding results. Assay optimization module 108 preferably comprises a database containing data relating to binding determined under different experimental conditions for the protein of interest, which in this Example is an IgG antibody. Assay optimization module 108 preferably receives a file containing optimized experimental results for other proteins of a particular type, such as IgG antibodies for example. The file preferably includes data relating to:

Calibration Standard

Desired sample in various exposure condition matrix (different exposure solution concentration and different exposure temperature)

Reference Sample

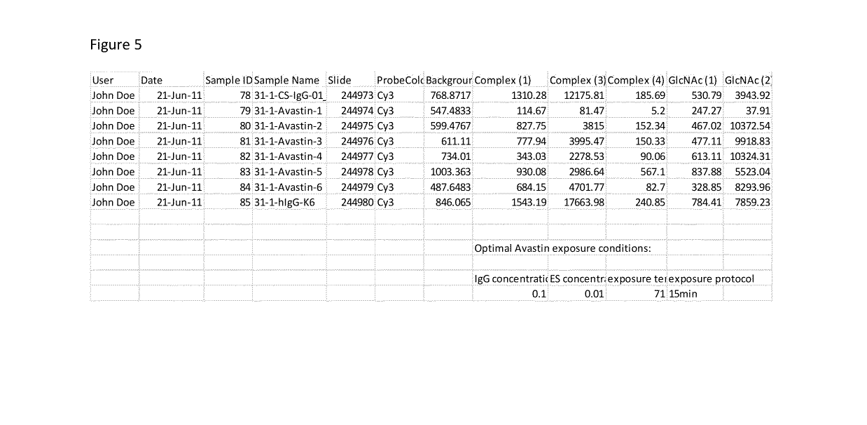

A non-limiting example of an experiment layout for such experiment (see FIG. 5 for example):

FIG. 5 shows an example of an optimization/exposure calibration experiment results. This experiment is preferably performed once, to determine optimum conditions for future experiments with the sample. The table below shows the experimental layout (the required slides and samples with the relevant tested exposure conditions). When the experiment is done, a set of results/glycoanalysis is generated by the system with additional information such as user name, date etc. as seen in FIG. 5. The report seen in FIG. 5 is uploaded to the assay optimization software module for analysis and the system generates a report indicating the optimal conditions for running the assay in the future.

| Pad/Slide No. 1 | Pad/slide | Pad/Slide | Pad/Slide | Pad/Slide | Pad/Slide | Pad/Slide | Pad/Slide No. 8 |
|---|---|---|---|---|---|---|---|
| (top pad/1^{st} slide) | No. 2 | No. 3 | No. 4 | No. 5 | No. 6 | No. 7 | (lower pad) |
| medium exposure | No | medium exposure | medium exposure | low exposure | High exposure | High exposure | High exposure |
| solution | exposure | solution | solution | solution | solution | solution | solution |
| concentration in % | treatment | concentration | concentration | concentration | concentration % | concentration % | concentration % |
| (low temp ° C.) | Tested | in % | in % | in % | (low temp ° C.) | (high temp ° C.) | (low temp ° C.) |
| Calibration standard | IgG | (low temp ° C.) | (low temp ° C.) | (low temp ° C.) | Tested IgG | Tested IgG | Reference sample |
| (Provided) | | Tested IgG | Tested IgG | Tested IgG | | | (Provided) |

Each pad or slide relates to a type of material and or combination of material and specific sample exposure conditions and hence to an experimental group to create an experiment condition set. For example, for one pad slides (such as slide number 2 for example), there is one material on each slide, with specific exposure conditions (temperature, exposure solution concentration and exposure time). Shared calibration standards are provided between slides for enabling corrections to the data.

Multi-pad slides feature one substrate, such as a one planar substrate, but with multiple sub-areas. For such slides, typically the first pad is a calibration standard. Optionally separate pads will feature samples, reference standards and controls. More than one probe may be provided on the same slide or pad/subarea with different detection agents (such as for example colorimetric agents having different colors).

In addition, if available, assay optimization module 108 may also optionally receive comparative HPLC (high pressure liquid chromatography) data for the same sample protein and at least one of the saccharide binding proteins present in array 102; the data is optionally obtained according to one or more methods that are known in the art.

Assay optimization module 108 then compares all available reference data and actual data obtained with the sample protein, to determine the most suitable assay conditions by calculation of optimal lectin activity, measurement of lectins that generate signals only when the sample is exposed and by calculating the slide background value compared to a calculated baseline defined based on an experimentally determined database created using a wide range of sample types.

Non-limiting examples of such parameters include binding time, temperature and exposure solution concentration value of buffer for incubating the sample protein with the saccharide binding agents; lectin (saccharide binding agent) signal, signal ratio, a set of designated lectins that react when the exposure was not optimal, background value and background value compared to the calibration standard background; ES (exposure solution) concentration in the sample buffer (e.g. 0.01% of the sample buffer volume; and sample protein concentration. Assay optimization module 108 also specifically determines whether the IgG sample antibody protein has Fab glycosylation or O-link glycosylation; if so, then specific optimization conditions are implemented. The user is warned of the presence of such glycosylation, which may negatively affect the accuracy of the results. Optionally one or more of the following additional parameters are optimized:
Sample concentration
Slide format
Assay format
Interpretation algorithm (script)

Optimization is performed in order to customize the assay to a specific mAb (antibody) and allow maximal exposure of Fc (immune molecule) related glycans. The optimization includes calibration of at least three parameters: Exposure Solution concentration, Temperature and incubation time of exposure pre-treatment, to expose glycan to the arrayed lectins.

Exposure solution may optionally comprise any ingredients that are suitable for exposing the unexposed glycan structures, which would otherwise be blocked. Optionally and preferably the solution features a detergent; more preferably the detergent is selected from the group consisting cholate, deoxycholate, C₁₆TAB, LysoPC, CHAPS, Zwittergent, SDS, Octylglucoside, Digitonin, Lubrol, C₁₂E₈, Triton X-100, Nonidet P-40, and Tween-80, all at percentages that are known in the art for unfolding proteins.

The exposure conditions may slightly vary from one monoclonal antibody to another due to differences in the protein sequence, which may affect the antibody biochemical and structural properties.

The condition matrix to be checked may optionally include:
Exposure solution concentration optimization: 0.001 to 1%
Temperature optimization: 50-80° C.
Time optimization of pre-treatment incubation: 1 minute to 1 hour

The selection of optimal conditions is automatically performed by a software method as described herein.

### Example 2

### Multi Concentration Arraying Format

The multi concentration array format relates to improvements in the physical array itself, to obtain more optimal binding results. This new grid format features the use of a concentration curve printed for each lectin (or antibody—saccharide binding agent) on the array compared to one concentration in previous array types.

The multi concentration format was developed in order to improve the system reproducibility and generate more accurate signals. Without wishing to be limited to a closed list, the multi concentration array format also provides at least the below improvements:
Higher repeatability in the fingerprint and interpretation level
Improved and more reliable way to define base line in some cases of non-specific binding.
multi concentration format solves the various problems such as “lectin zero point”, reproducibility and sample concentration, which can be significant for high backgrounds or low signals. Solution of this problem enables the amount of saccharide binding agent printed on the array to be reduced.

The multi concentration format defines a multiple spot per binding agent including concentration curve for each saccharide binding agent, such as a lectin and hence requires more spots on each array. To achieve this goal the arraying format was modified and includes additional spots per lectin. Also the plate format was changed to include all the concentrations required and additional labeled spots for improved automatic image analysis. Adding more spots may reduce the ability of an automatic image analysis software to accurately detect the spots on the array and locate a virtual measurement grid (to set the spot value and calculate the fingerprint). To improve the image analysis capabilities, additional labeled spots may be provided, including a pre-labeled protein, which provide a high signal during the processed array scanning

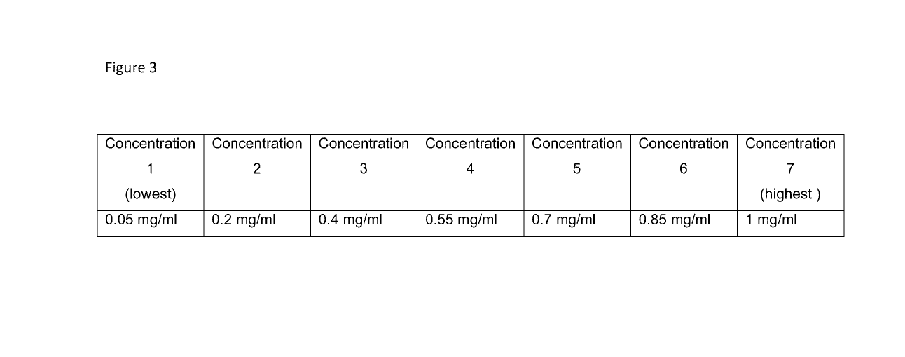

As noted above, the multi concentration format defines a concentration curve for each saccharide binding agent, such as a lectin for example. In this non-limiting Example, concentration curves are given for the amount of lectin printed on the solid substrate of the array over a plurality of spots. Preferably, there are a plurality of different spots, associated with different protein concentrations, for each lectin (saccharide binding agent) on the array substrate. Optionally, the concentration range is from 0.01 mg/ml to 10 mg/ml; preferably the range is 0.038 mg/ml to 3.5 mg/ml and more preferably from 0.05 mg/ml to 1 mg/ml (given as amount of lectin in milligrams per milliliter of the spotting solution). For example the concentrations of the lectin ConA are given in FIG. 3, with 7 different concentrations (and hence 7 different spots on the physical substrate, each with the amount of lectin given in the table),

The multi-concentration printing format is used to calculate a curve of signals for each lectin on the slide. As noted above, the system 100 preferably automatically determines which concentrations are acceptable; if there are problems with the signal and linearity, up to 2 concentration points in the curve (out of total number of concentrations) may optionally be ignored. The quality of the calculated curve and signals determine the value of the presented fingerprint errors. It is expected that the binding signal will be linear over at least 5 of the total number of lectin concentrations; this parameter is one of the parameters tested by assay optimization module 108 and is one of the criteria for selecting a particular set of experimental conditions, as described with regard to Example 1.

The multi concentration format may optionally be prepared on an array, which as previously noted is preferably a planar substrate, through contact or non-contact printing. As its name suggests, contact printing relates to touching a solid device, such as a pin head, coated with a solution containing a saccharide binding agent, to the surface of the planar substrate. Non-contact printing may for example optionally involve spraying or other distribution means of a solution containing a saccharide binding agent, such that the agent becomes deposited on the planar substrate.

For contact printing the spot size diameter preferably varies from 0.05 mm to 75 mm. For non-contact printing, the spot size diameter preferably varies from 80 microns to 2500 microns.

By “diameter” it is meant the longest axis (dimension) in a spot; the spots do not need to be circular or symmetrical about their axes.

In terms of the amount of sample protein for each spot, for IgG proteins, the recommended IgG amount is in the range of 1.8-12 μg. The recommended IgG concentration is in a range of from 0.001 micro-molar to 100 micro-molar; 0.1 μM is particularly preferred.

### Example 3

### QC Monitors and Calibration

QC (quality control) monitors and calibration relate to improvements in the assay system.

QC monitors identify technical problems in the wet (slide processing) and dry (scanning) phases) of the workflow of system 100. The main issues examined are the slide quality, scanning quality, correlation between duplicates (if available) and problems during the image analysis step. The user is provided with numeric scores (in a scale of 0-1 for technical quality and −100 to −200 for image analysis grid alignment quality) for slides and samples in the experiment. More details about the internal components of each score are provided in the report provided for every sample.

### Computational Phases:

The computational phases of the algorithm start with the testing of each of the spots on the slide; each spot receives a score according to its homogeneity and background level. Some spots can be excluded in that process. The entire slide is then scored according to its average spot quality, the correlation between its two identical sub blocks, and other technical parameters. The next phase is to test correlation between duplicate slides (two sample slides, or two control slides). If the duplicate correlation is bad, the system automatically selects the better slides as the basis of the computation.

The following table summarizes the main monitors used for computing the slide score; it should be noted that the below table is an example for one slide type and can be different for additional slide formats and/or surface types:

| Type of monitor | Criteria |
|---|---|
| The surface of the | Average BG |
| slide (background | BG Coefficient of Variance |
| or BG) | Percent of Extreme Points |
| | Zero BG |
| | Black Holes |
| | (BG > Density) |
| Control points | Percent of High PBS Spots |
| | Percent of High BSA Spots |
| | Percent of Low BSA-Fitc Spots |
| Reproducibility | Intraslide Correlation |
| | Intraslide Slope |
| | Intraslide Intercept |
| Spot level | Percent of Spots with high CV |

A non-limiting list of QC monitors is provided below:

Spots Evaluation—
In this process, spots are evaluated by homogeneity of foreground, homogeneity of background, similarity between mean median density, level of saturation. Low quality spots are excluded from the normalization stage between duplicates.

Slide Validation—
In this process entire slide is evaluated and each slide receives a score based on its technical quality (based on background of the slide, control spots, intra slide reproducibility). A low quality slide is defined as a slide in which >=50% of the spots are low quality or there is low intra slide reproducibility or low inter-slide reproducibility. Low quality slides are excluded.

Evaluation of the Fit Algorithm—
The ‘fit’ algorithm is used for normalization between sample and control slides. In this process the quality of the fit algorithm is evaluated. (Based on variability of the slope coefficient, comparison of signal to zero for lectins that are used for fit, % of signals>0)

Evaluation of Sample Vs. Control—
In this process the combined signal of the experiment is tested. (Check that the combined signal is higher than zero for lectins that aren't used for fit, % of lectins that produces combined signals>0)

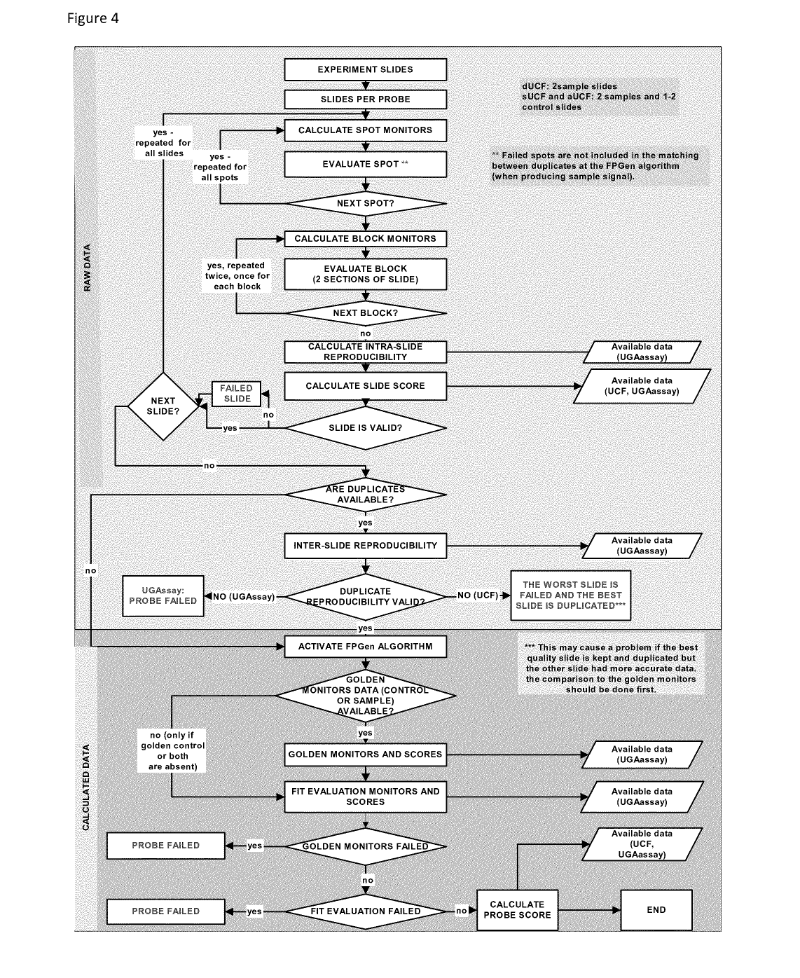

FIG. 4 shows a flowchart for implementation of the QC monitors within system 100 of FIG. 2. As shown in FIG. 4, which relates to the solid array surfaces as “slides” as a non-limiting example only, the initial part of the process relates to “raw data”. In this part of the process, which is repeated for all slides as shown, all experimental and control spots (locations on the array) are evaluated for their signal. The control spots include spot monitors (controls related to a particular spot) and block monitors (controls related to a slide or to a section of a slide). After these spots have been evaluated, the intra-slide quality (reproducibility within a slide) is evaluated. Each slide is then scored; the slide is considered either to have passed (i.e. to have data of sufficiently high quality) or to have failed. If a duplicate slide is available, it is also evaluated.

Next, in the part of the process related to “calculated data”, “golden monitors” (previously determined data, having expected result) are evaluated and are scored. If their scores are sufficiently high, then the experimental data results may be analyzed accordingly as described herein. Otherwise, the experimental data is rejected, as the underlying assay is determined to be unreliable.

Calibration Standard Algorithm

Calibration protein was added to the assay for a standard in this non-limiting example. The calibration standard protein serves as a calibration tool for the analysis. For each assay a specific calibration standard protein is chosen (some wider assay types such as a generic IgG assay may include a generic calibration standard). The calibration standard protein is a variant of the type of glycoprotein for which the assay is built (for example, as in Example 1, the standard protein may be a known IgG antibody with known glycosylation properties). The calibration standard protein is included in every experiment and used to calibrate the lectin reactivity according to a “golden” standard” (the expected fingerprint, or saccharide binding agent binding profile, of the calibration standard protein). The calculation is based on normalization between the real fingerprint resulting in an experiment and the “golden” fingerprint, and correcting the outlier reactions that result from experimental conditions. This procedure enables quantified and accurate glycoanalysis.

Assay data analyzer 106 of system 100, for example, may optionally compare the actual with expected binding results according to class of protein. For example the “golden” standard or binding fingerprint may optionally be determined for IgG antibodies or other classes of proteins, and then used for a comparison to actual binding results obtained with a particular calibration protein. Preferably however the golden standard fingerprint is obtained for the same calibration protein that is printed on the solid substrate of the array 102. A non-limiting exemplary method is described below:

Fingerprint comparison: compare fingerprint of the CS (calibration sample) sample to the golden fingerprint (which is a “gold” standard set of results):
Normalize to average gold and calibration standard samples
Perform T test, reject outliers, and compute regression on non-outlier lectins (saccharide binding agents).
If in the T test, ⅓ or more lectins were rejected—CS is disqualified
If the resulting slope <X or >Y—CS is disqualified
If the resulting R²<x—CS is disqualified

Furthermore, optionally a correction factor is obtained from the above calibration method as follows for each saccharide binding protein: CS value/golden fingerprint value. This correction factor is preferably applied to binding data of actual sample proteins by assay data analyzer 106 for example.

### Example 4

### Out of Assay Flag for the Array and the System

The out of assay flag was developed to reduces errors and detect problems in the assay by flagging “outlier” or unusual results. For sample variants whose glycosylation pattern deviates considerably outside the bounds of the data generated during an assay development, out of assay flag module 107 of the system of FIG. 2 as previously described generates a message that warns the user that a problem has occurred. During specific assay development for a given glycoprotein sample, a set of glycosylation variants that are likely to occur in this sample is created. The interpretation script is developed, optimized and tested on this set. One can expect high-accuracy interpretations for samples whose glycosylation pattern is within the domain defined by these variants. For variants whose glycosylation pattern deviates considerably outside the bounds of this domain, no such guarantee can be provided. Still, the system will identify that the sample's glycosylation pattern is considerably different from the original samples and their derivates; a warning message (“out of assay flag”) to this effect is provided, and may for example optionally be displayed to the user or sent to the experiment log file, or otherwise recorded.

Without wishing to be limited by a closed list, some non-limiting examples of situations that may trigger the out of assay flag include:

1. appearance of new variant that does not reflect actual expected fingerprint (for example detection of a saccharide or polysaccharide motif that was not expected in a specific glycoprotein):

a. a predicted variant that could not be produced bio chemically (such as different distributions of bi- vs. tri-antennae)

b. an unpredicted variant which contains an unusual glycosylation structure

(unusual in the context of the specific sample)

2. unusual fingerprint of an already tested sample protein.

3. A user error, such as the wrong sample used.

4. Failure of the assay itself; for example, low to no signals from the assay

The “out of assay” warning is based on a few simple knowledge-based rules. These rules are the result of studying the behavior of the specific sample variants combined with accumulating knowledge on the lectins. In most assays the basic rules that define the fingerprint as “out of assay” are:

1. significant change in the ratio between the bi vs. tri_tetra antennary structures from what is expected (this rule is based on the complex vs. the mannose lectin binding motifs)

2. appearance of a new unit (saccharide/polysaccharide motif). In most cases such units are detected as yes/no and upon detection an “out of assay” warning is in order (most common are High Mannose structures and Terminal GalNAc unit, as in many cases their appearance in binding results is not expected)

3. unexpectedly low signals for lectins that are expected to react with a minimum value, based for example upon the type or category of sample protein, or previous results with the same sample protein.

Testing was performed by running the script on the sample benchmarks. The results were that in most cases the fingerprints were within the assay boundaries, with a few “out of assay” fingerprints. In most cases this warning was reasonable.

### Example 5

### IgG Experiment

The above system was tested for one array type (one pad slides) using an exemplary IgG antibody, Avastin. The results of the experiment are shown in FIG. 5. Briefly, Avastin was incubated with a number of different lectins as saccharide binding agents (Complex (1), Complex (3), Complex (4), GlcNAc (1) and GlcNAc (2) as shown), along with background samples and with different sample exposure condition parameters to determine the optimal conditions for the sample assay (as shown in FIG. 5). Different sample names under the “sample name” relate to standards (samples 78 and 85) and to Avastin itself (samples 79-84; given different sample names to indicate changes in conditions etc). Following the “background” column, the five columns to the right relate to different lectins, with different lectin names given.

The optimal conditions for this protein were as follows (given as optimal Avastin exposure conditions in the lower right corner): IgG concentration (micro-molar): 0.1; exposure solution concentration—0.01% (relates to the amount of detergent in the incubation solution, for exposing certain saccharide/polysaccharide motifs as previously described); exposure temperature was 71 C; and the best time for exposure was found to be 15 minutes. It should be noted that this is a non-limiting example only; using a different array and/or assay type may require different conditions.

While the invention has been described with respect to a limited number of embodiments, it will be appreciated that many variations, modifications and other applications of the invention may be made.

What is claimed is:

1. A glycoanalysis array, comprising a planar substrate and a plurality of saccharide binding agents present on a surface of said substrate at a plurality of predetermined locations, each of said plurality of saccharide binding agents being present at a plurality of separate predetermined locations on said surface, wherein said plurality of separate predetermined locations relates to a plurality of concentrations of said saccharide binding agent at said locations in a concentration curve; said planar substrate being adapted for being contacted with a sample comprising a glycoprotein, such that said glycoprotein binds specifically to at least one saccharide binding agent and forms a detectable binding complex, such that a baseline for non-specific binding is determined according to said concentration curve.

2. A glycoanalysis array, comprising:
a planar substrate; and
a plurality of contact portions present on a surface of said planar substrate at a plurality of predetermined locations, wherein a plurality of saccharide binding agents are provided in the plurality of contact portions, the plurality of saccharide binding agents being provided in predetermined locations on said planar substrate, wherein each of said plurality of saccharide binding agents is present at a plurality of separate predetermined locations on said surface, wherein said plurality of separate predetermined locations are provided with a plurality of concentrations of said saccharide binding agent in a concentration curve; said planar substrate is provided to contact with a sample comprising a glycoprotein, such that said glycoprotein binds specifically to at least one saccharide binding agent at the plurality of contact portions and forms a detectable binding complex, such that a baseline for non-specific binding is determined according to said concentration curve.

3. The array of claim 1, wherein said detectable binding complex is detectable through a binding signal and wherein said binding signal is linear over at least five of the total number of saccharide binding agent concentrations in said concentration curve.

4. The array of claim 1, wherein said planar substrate features indentations or wells.

5. The array of claim 4, wherein said planar substrate comprises at least one of a membrane, glass or plastic.

6. The array of claim 5, wherein said planar substrate is derivatized.

7. The array of claim 3, wherein said array further comprises a plurality of labeled predetermined locations on said planar substrate for providing a high signal for supporting image analysis of said array after being contacted with said sample.

8. The array of claim 7, wherein said array further comprises a plurality of saccharide binding agents as calibration standards in predetermined locations on said planar substrate.

9. The array of claim 8, wherein said planar substrate is divided to a plurality of pads and wherein each pad comprises a separate plurality of calibration standards.

10. The array of claim 1, wherein said saccharide binding agents comprise lectins or antibodies, or modified components or a combination thereof.

11. The array of claim 10, wherein a concentration range for each lectin on said substrate is from 0.01 mg/ml to 10 mg/ml.

12. The array of claim 11, wherein said concentration range is 0.001 mg/ml to 10 mg/ml.

13. The array of claim 12, wherein said concentration range is from 0.01 mg/ml to 5 mg/ml.

14. The array of claim 11, wherein each location has a spot size diameter and wherein said spot size diameter is in a range of from 0.05 mm to 75 mm.

15. The array of claim 11, wherein each location has a spot size diameter and wherein said spot size diameter is in a range of from 80 microns to 2500 microns.

16. The array of claim 14, wherein said glycoprotein comprises an IgG antibody or fragment.

17. The array of claim 16, wherein said IgG amount is in the range of 1.8-12 μg per location.

18. The array of claim 17, wherein said IgG concentration is in a range of from 0.001 micro-molar to 100 micro-molar in a solution for contacting said IgG antibody or fragment to said saccharide binding agents.

19. The array of claim 18, wherein said solution comprises a detergent for unfolding said IgG antibody or fragment and exposing at least one glycan.

20. The array of claim 3, wherein said planar substrate features indentations or wells.

21. The array of claim 3, wherein said saccharide binding agents comprise lectins or antibodies, or modified components or a combination thereof.

22. An assay system for performing glycosylation assays with a plurality of arrays according to claim 1, comprising a kit for performing said glycosylation assay, a detector for detecting said detectable binding complex through a binding signal to obtain binding data and an assay optimization module for comparing reference data and said binding data obtained with the sample glycoprotein, to determine suitable assay conditions by calculation of optimal lectin activity, measurement of lectins that generate signals only with the sample glycoprotein and calculation of the slide background value compared to a calculated baseline defined based on an experimentally determined database created using a wide range of sample types.

23. An assay system for performing glycosylation assays, comprising:
a plurality of arrays, each of the plurality of arrays comprising
a planar substrate; and
a plurality of contact portions present on a surface of said planar substrate at a plurality of predetermined locations, a plurality of saccharide binding agents are provided in the plurality of contact portions, the plurality of saccharide binding agents provided in predetermined locations on said planar substrate, each of said plurality of saccharide binding agents is present at a plurality of separate predetermined locations on said surface, wherein said plurality of separate predetermined locations are provided with a plurality of concentrations of said saccharide binding agent in a concentration curve; said planar substrate is provided to contact with a sample comprising a glycoprotein, such that said glycoprotein binds specifically to at least one saccharide binding agent at the plurality of contact portions and forms a detectable binding complex, such that a baseline for non-specific binding is determined according to said concentration curve;
an assay performance module, wherein the plurality of arrays are connected to the assay performance module, or contained in the assay performance module;
a detector connected to said assay performance module, wherein binding of the saccharide binding agents to the sample protein is detected through said detector;
an assay data analyzer, which is in communication with said detector or connected to said detector;
an out of assay flag module connected to said assay data analyzer or in communication with said assay data analyzer; and
an assay optimization module, connected to said assay data analyzer or in communication with said assay data analyzer.

24. The assay system of claim 23, wherein said assay optimization module further determines suitable assay conditions according to one or more of the following parameters: binding time, temperature and pH value of buffer for incubating the sample protein with the saccharide binding agents; saccharide binding agent signal, signal ratio, a set of designated saccharide binding agents that react when the exposure was not optimal, background value and background value compared to the calibration standard background; detergent concentration in the sample buffer; and sample protein concentration.

25. The assay system of claim 24, wherein said assay optimization module optimizes sample glycoprotein concentration, substrate format and assay format.

26. The assay system of claim 25, wherein said sample glycoprotein comprises an IgG antibody and wherein said assay optimization module determines whether the IgG sample antibody has Fab glycosylation or O-link glycosylation, such that specific optimization conditions are implemented and a warning is issued regarding the presence of such glycosylation.

27. The assay system of claim 26, wherein said assay optimization module determines an amount of exposure solution to be applied to said sample glycoprotein, wherein said exposure solution features a detergent selected from the group consisting of SDS (sodium dodecyl sulfate), cholate, deoxycholate, C16TAB, LysoPC, CHAPS, Zwittergent, Octylglucoside, Digitonin, Lubrol, C12E8, Triton X-100, Nonidet P-40, and Tween-80, at a percentage known in the art for unfolding proteins.

28. The assay system of claim 27, wherein said assay optimization module determines a condition matrix relating to one or more of the following: Exposure solution concentration optimization: 0.001 to 1%, Temperature optimization: 50-80° C., Time optimization of pre-treatment incubation: 1 minute to 1.

29. The assay system of claim 22, further comprising at least one QC (quality control) monitor selected from the group consisting of spots evaluation by homogeneity of foreground, homogeneity of background, similarity between mean median density, level of saturation; array validation to evaluate a quality of an entire planar substrate according to background of the array, control spots, intra array reproducibility; evaluation of normalization between sample and calibration locations; determination of overall array signal.

30. The assay system of claim 22, further comprising a calibration protein for being applied to a plurality of predetermined locations containing said saccharide binding agents as a calibration sample, to calibrate saccharide binding agent reactivity according to a golden fingerprint standard by said assay optimization module.

31. The assay system of claim 30, wherein said assay optimization module determines an out of assay flag according to a comparison of binding signals from said sample glycoprotein to said calibration sample.

32. The assay system of claim 31, wherein said assay optimization module issues a warning according to said out of assay flag.

33. The array of claim 2, wherein said contact portion is selected from grooves, indentations or wells having a circular, elliptic or rectangular shape.

34. The assay system of claim 23, wherein said contact portion is selected from grooves, indentations or wells having a circular, elliptic or rectangular shape.

35. The assay system of claim 23, wherein said array is inserted into or provided to said assay performance module.

36. The assay system of claim 23, wherein said detector is combined with or separated from said assay performance module.

37. The assay system of claim 23, wherein said detector is combined with or separate from said assay data analyzer.

38. The assay system of claim 23, wherein said assay optimization module comprises a database containing data relating to binding determined under different experimental conditions for the protein of interest.

39. The assay system of claim 23, wherein said assay optimization module receives a file containing optimized experimental results for other proteins of a particular type.
